(19) United States
(12) Patent Application Publication
(10) Pub. No.: US 2006/0271039 A1
(43) Pub. Date: Nov. 30, 2006

(54) Device and method for the repositioning of prolapsed haemorrhoids

(76) Inventor: Norbert Vetter, Grand River (CA)

Correspondence Address: HEDMAN & COSTIGAN P.C., 1185 AVENUE OF THE AMERICAS, NEW YORK, NY, 10036, US

(21) Appl. No.: 11/436,931

(22) Filed: May 18, 2006

(30) Foreign Application Priority Data
May 19, 2005 (DE) 10 2005 022 998.0
Dec. 23, 2005 (DE) 20 2005 020 275.4

(51) Int. Cl. A61B 18/18

(52) U.S. Cl. 606/45

(57) ABSTRACT

The invention relates to a device and a method for the repositioning of prolapsed haemorrhoids with a press head arranged on a base.

### BACKGROUND OF THE INVENTION

The invention relates to a device and a method for the repositioning of prolapsed haemorrhoids with a press head arranged on a base.

The corpus cavernosum recti, as a cushion comprising blood vessels, smooth muscle fibres, elastic fibres and connective tissue in the anal duct, has the physiological property of being able to expand and contract, and therefore serves as a “fine seal” for fine continence. Considering that a corpus cavernosum recti is present in each of us, the term “haemorrhoids” is largely confined to cases in which pathogenic symptoms result from them. These include, for example, enlargement, where the cushion slides down below the anoderm and into the lower sections of the anal duct, thereby giving rise to a displacement of the rectal mucous membrane into the anal duct, with corresponding disturbance of sensitivity and bleeding from this cushion. The occasionally bleeding haemorrhoids are also described in the literature as mucous membrane haemorrhoids (definition according to Zetkin/Schaldach).

Haemorrhoids are varicose extensions of the vein network in the anorectal region, the condition of the wall normally being chronically inflamed or scarred. The external haemorrhoids project into the form of nodes outside the anus. This projection of the haemorrhoids from the anus is known as prolapse.

According to the definition of Schütz/Rotschuh the pathogenic extension of the cushion-like knot of veins located just above the anus at the end of the mucous membrane, which normally serves as a tight occlusion, is termed haemorrhoid(s). According to general medical understanding haemorrhoids are therefore cushions (cushion-like knots of veins) consisting of small arteries and veins and arteriovenous anastomoses which, when enlarged and filled to a high degree, drop or prolapse into the anal duct and may bleed. Occasionally prolapsed haemorrhoids may harden due to blood clotting.

The symptoms of haemorrhoids are clinically divided into different grades. For instance, painless bleeding with no or only slight enlargement of the corpus cavernosum recti is described as Grade 1, considerable enlargement with bleeding and haemorrhoids visible in the anal duct when straining is described as Grade 2, prolapse of the haemorrhoids in the anal duct when straining or during bowel movement, with spontaneous reposition, is described as Grade 3, and prolapse of the haemorrhoids, which can only be repositioned manually, is described as Grade 4.

The phenomenon of outward sagging of haemorrhoids is known as haemorrhoid prolapse or anal prolapse. This prolapse gives rise to unpleasant feelings of pressure and pain. If the mucous membrane is inflamed in the region of the prolapsed haemorrhoids, a corresponding degree of bleeding may take place, frequently occurring during or after a bowel movement or even physical effort and in most cases stopping very quickly after the haemorrhoids are repositioned.

The prolapse that occurs during bowel movement result in irritation of the anoderm and the rectal mucous membrane extending into the lower anal duct. This gives rise to micro-lesions and bleeding from the haemorrhoids, with subsequent scarring. If such a condition remains untreated the submucous structures will also be scarred, with fixation of the submucous structures on the caudal sections of the musculus sphincter ani internus and in the long term an anal prolapse that cannot be repositioned developing from an anal prolapse that could be repositioned until that point. A distinction is made here between the musculus sphincter ani internus (internal anal compressor muscle) and the musculus sphincter ani externus (outer anal compressor muscle).

The more frequently the haemorrhoids project and the longer the filled prolapsed haemorrhoids remain untreated in the projected condition, the greater the risk that the prolapsed haemorrhoids will no longer be able to reconstitute themselves spontaneously. The haemorrhoids fill abnormally with blood at ever shorter intervals and also project from the compressor opening without much physical effort.

Operations to remove haemorrhoids are not always without problems because in unfavourable circumstances (lesion of the corpus cavemosum recti or even the compressor muscles), and as a result of scarring, the sealing mechanism may be impaired. In isolated cases it will then no longer be possible to distinguish between gas and liquid intestinal content. In stage 4, under certain circumstances, the anal region may have to be reconstructed, depending on the operation method. Haemorrhoid operations provide no guarantee that haemorrhoids will not again expand and prolapse abnormally.

Abnormally sagging haemorrhoids also fill up, according to the symptoms, after light physical effort or in unfavourable physical positions, at work or when engaged in sport, as well as in straining whilst emptying the bowels. In the latter case the anal region with the prolapsed haemorrhoids should be thoroughly cleaned preferably with water only because there will an increasing amount of sagging skin folds in the anal region between which stools will lodge under certain circumstances. This cached stool may result in unpleasant reactions and inflammations, as well as irritation of the mucous membrane. After repositioning the haemorrhoids gentle washing lotion can be used on the outer parts of the anal region.

Prolapsed haemorrhoids substantially impair the capacity to work and may also result in absence from work. General well-being is greatly limited. In the case of inflammations cortisone preparations are often prescribed, which should be used under medical supervision.

### SUMMARY OF THE INVENTION

The object of the invention is to provide a device with which prolapsed haemorrhoids can be repositioned simply, quickly and hygienically in the anal duct.

The established object is achieved by means of a device or arrangement for repositioning haemorrhoids according to independent claims. Preferred embodiments are the subject of the dependent claims or are described below.

### DETAILED DESCRIPTION OF THE INVENTION

The repositioner is designed, in terms of length and cross-sectional area, and particularly in terms of length, so that the press head cannot be introduced rectally into the anal duct when used correctly.

Haemorrhoid treatment rods which act as anal expanders are known from the prior art. These rods are long and pointed. The anal expander is used, for example, for prae- and post-operative treatments, but it also has, in principle, a different function and form. The anal expander is so long that it is introduced into the anal duct to widen the anal duct but not to reposition haemorrhoids into the anal duct. It does not have the flattened front side which forms a kind of press head and which is an important precondition for rapid repositioning of projected haemorrhoids.

The function of the device according to the invention, unlike an anal expander, consists in particular in that prolapsed haemorrhoids that have filled with blood are pushed back behind the compressor muscle aperture and are emptied by the variable contraction of the compressor muscle opening to a more or less normal degree. The peripheral edge of the press head is of particular importance here.

When the prolapsed haemorrhoids are repositioned the uncomfortable feelings of pressure or pressure pain quickly recede. The repositioner should preferably be used immediately after a bowel movement and after prior thorough cleaning of the anus where the relevant symptoms occur.

It is highly advantageous to use the repositioner even if the symptoms are only mild. If it is applied in good time further prolapse can generally be avoided or existing prolapse reduced, and their further prolapsing prevented in the initial stages of the disease.

It has been shown that when the device according to the invention is used, operations can be avoided and a condition is reached in which there is no longer a requirement for immediate operative intervention. It also helps avoid a situation where cortisone preparations have to be regularly used.

The device according to the invention can advantageously be deep drawn from one piece of material, with press head and base, in the form of a base plate. The device can therefore be manufactured very economically. The deep drawing material may consist of plastic or metal, but the device according to the invention may also consist of wood. For hygienic reasons, however, plastic and metal are preferable as they are much easier to clean. The material should also be tolerated by the skin.

A hollow design is preferred but not required and the invention may be provided in another design where the device consists of solid material.

The plinth, as a support for the press head on the base of the stand, may in principle be of any shape. The plinth must not have a rectilinear wall in the shape of a truncated cone, and essentially it is the press head alone that performs the function constituting the object of the invention. It is also preferable for the plinth to be designed with a round or oval cross-section (with the press head viewed in elevation). If necessary the plinth and the press head may also be polygonal in elevation (more than 4 corners). The press head exhibits a closed press head front face between the peripheral edges. For application method 2 the upper cross section of the plinth may as well have the form of a square or a triangle with rounded corners.

According to a further embodiment of the invention provision is made for the contour of the stand base to be designed in the form of a base plate, and for this to be round, angular or oval if necessary. Its function consists in guaranteeing a tilt resistant stand surface for the device so that the device can be placed on a surface and the repositioner, when used, can be reliably guided along the anus without tilting (generally on until the press head reaches the compressor muscle aparature).

The base or base plate also performs the function of limiting the advance of the press head when the buttocks are positioned on the base or on the upper base area, and if the buttocks are placed on a surface, including this surface.

If the device is hollow a bottom cover may be provided in the region of the base plate. If the device is sealed with the bottom cover, a small tube of haemorrhoid ointment, a cleaning cloth, a sealable bag and/or a fingerstall may be stored in the hollow. On journeys, at work and in sports this is very useful in dealing with the haemorrhoids.

According to a further embodiment of the invention the bottom cover may be inserted in the base plate or placed over the base plate.

According to a further embodiment of the invention provisions is made for the inside of the repositioner, with integrated press head, to be filled with a cooling core or a coolant. Cooling of the anal region may considerably relieve the complaints caused by the haemorrhoids. In the case of repositioning, however, only a brief cooling action on the haemorrhoids may be expected, but the surrounding skin that may be irritated can be cooled in this way. However, such an embodiment is only provided if the repositioner is used directly, i.e. without inserted textile or clothing (Embodiment 2).

The press head is advantageously raised from the upper edge of the stand base or base plate by approx. 30 to 50 mm, preferably approx. 40 mm. In principle the repositioner with press head will generally have such a length that it does not penetrate the anal duct during the repositioning process but is able to develop the pressure required for repositioning. For this purpose the press head may maximum extend to a point immediately in front of the opening of the external compressor muscle.

What is important here is the action of the press head of the device, i.e. the front flattened region of the press head with which the projected haemorrhoids are repositioned. The repositioning is reinforced by the clothing since the press head diameter is increased by the clothing which also prevents the press head from penetrating the anus.

A further object of the invention is to provide a method for repositioning prolapsed haemorrhoids, wherein the device is placed with the press head on the clothing from the outside through the anal opening, the device stands on a stand surface and the anus is lowered onto the device without the press head or clothing on the press head penetrating the anal duct after lowering.

### BRIEF DESCRIPTION OF THE FIGURES

FIG. 1 shows a device for repositioning projected haemorrhoids with a repositioner in the shape of a truncated cone, with press head and base plate,

FIG. 2 shows the device according to FIG. 1, in a view rotated 90°,

FIG. 3 shows an elevation of the device with a possible embodiment of the base plate,

FIG. 4 shows a cover for sealing the cavity in the repositioner,

FIG. 5 shows the press head, with part of the plinth enlarged, and

FIG. 6 shows the anus, with the device guided along it, and with the clothing placed on the device.

### DESCRIPTION OF THE PREFERRED EMBODIMENTS OF THE INVENTION

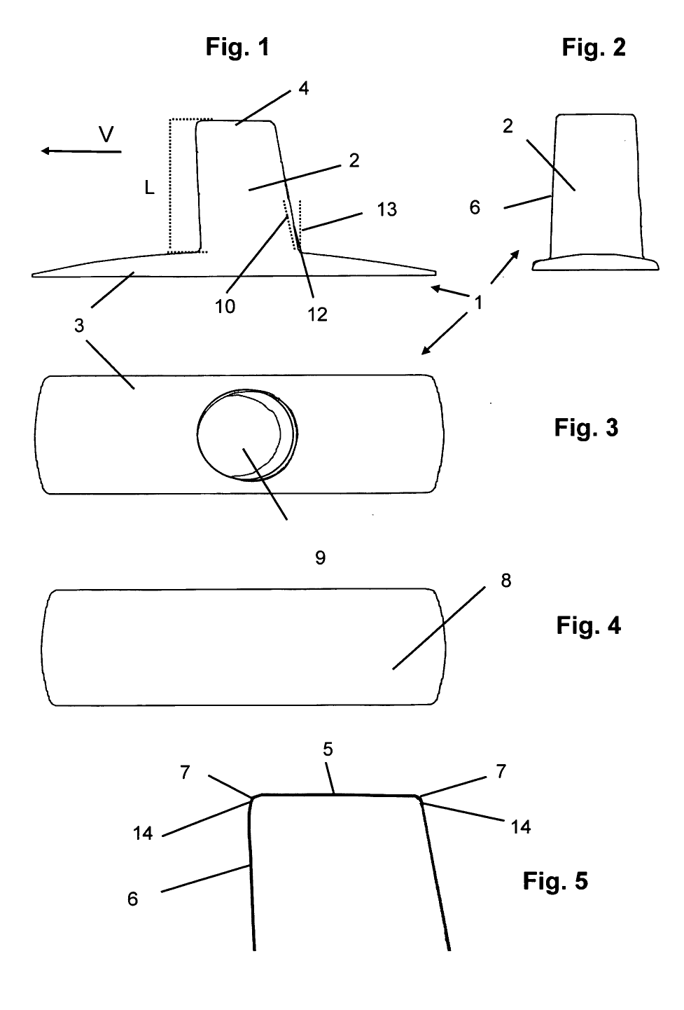

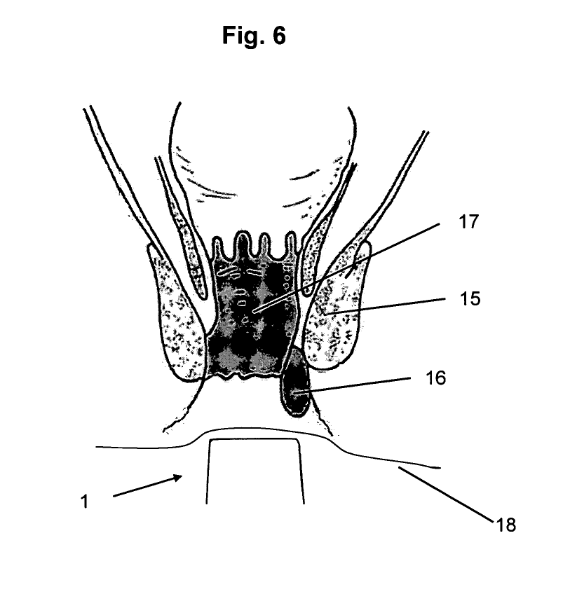

Device 1 shown in FIG. 1, for repositioning prolapsed haemorrhoids 16, consists of a plinth 2 with press head 4, wherein plinth 2 is designed in the shape of a truncated cone and is provided at one end with a base 3 in the form of a base plate. The plinth in the shape of a truncated cone runs conically in the upper head region as far as press head 4. On the front face 5 of the press head, press head 4 is flattened to form the truncated cone. The flattening is provided on plinth wall 6 with a peripheral edge 7, which may also be designed as a peripheral bead. The plinth in the shape of a truncated cone, with press head 4, is sealed with front press head face 5 in the region of press head 4.

Plinth wall 6 may be round or oval in cross-section. It is also possible to provide press head 4, in elevation, with a spherical or oval flattened shape. The press head surface may also be convex or concave as long as the press head still exhibits an external peripheral edge with horizontal surface sections.

As can be seen in the views shown in FIGS. 1 to 3, base plate 3 is designed in the form of a strip, but other shapes between angular to round and oval may of course also be used. Base plate 3 may also be detachable from plinth 2 if required.

Base plate 3 and truncated cone plinth 2, with integrated press head 4, may consist of solid material, but device 1 is preferably of hollow design. For economic production provision plinth 2, including press head 4 and base plate 3, may be manufactured in one piece. This can be achieved by injection moulding or deep drawing. Different plastics or metals, e.g. aluminium, may be considered as materials for deep drawing.

In this case use may be made of a cover 8, which may be inserted in the opening 9 at the rear side of device 1 or of plinth 2 or, as shown in FIG. 4, it may be placed or slid over the entire base plate 3. Cavity 9 that can be sealed in this way may be used in different manner. For example, a coolant, a cooling core or utensils for treating haemorrhoids may be accommodated in it.

Because of the slight inclination of the plinth 2 in the forward direction (V), possible tilting to the rear is prevented, whilst at the same time guaranteeing that it is possible to sit on the flattened surface of press head 4 and not obliquely on peripheral edge 7 (in the case of large angles 12).

Important is also the length L of plinth 2 with integrated press head 4. Length L of plinth 2 should be 30 to 50 mm, measured from the upper edge of base plate 3, preferably approx. 40 mm, to the highest point of the press head.

If the haemorrhoids prolapse and an uncomfortable pressure pain is felt, the device is placed so that it is stationary on a stable surface, in application method 1. The device stands on base plate 3, in which case the contact surface may, for example, be a chair, a bench or a toilet seat cover.

The experimental subject should find the correct position above the projecting haemorrhoids and carefully lower the anal region onto the device. Where the haemorrhoid nodes project considerably, it may be necessary to raise and lower the anal region several times. The positioning and repositioning of the haemorrhoids should take place slowly, and here the deliberate contraction of compressor musculature 15 may be helpful.

Length L of plinth 2 is relatively constant because the basic physical conditions for sitting do not differ substantially from one person to another. In people who are considerably overweight, a model with a longer plinth 2 may be necessary under certain circumstances, or a model may be used with an adjustable plinth length.

The device is designed so that press head 4 is not introduced into the anal duct. However, a certain repositioning pressure must be exerted on the anal region.

Arrow V (front) indicates the alignment of the body to the device. The arrow direction corresponds to the direction of vision of the experimental subject when using the device in the seated position.

According to application method 1 the clothing should be kept on during use. The press head is in this case applied simply to worn clothing 18. The time of application depends on the stage of the disease and symptoms prevailing at the time. Haemorrhoids 16 are repositioned in anal duct 17 within a short space of time, approx. 10 to 30 seconds or shorter, depending on the patient. It is not necessary to disinfect the device in this application method. However, cleaning and disinfection is recommended when the device is used in a public building or institution.

In application method 2 there is direct skin contact. In the hospital or at home the device can also be cooled, in which case it must be possible to seal press head 4 with a cover 8. The cooling core should not be cooled to a temperature of below 5° C., to avoid damage to the skin. Thorough cleaning and disinfection of the device is required in this case. Direct contact of the device with the skin is required to maintain the advantages of fast acting refrigeration. Moreover, the application corresponds to that in application method 1, apart from the fact that a clothing is not worn here, but a cloth is applied instead. In application method 2 the device must be thoroughly cleaned and disinfected before and after treatment. Application method 1 is preferred to method 2.

1. A device for repositioning haemorrhoids, comprising a base for stable positioning on an essentially planar stand surface, and a press head supported by a plinth and spaced away from the base, wherein
the press head comprises a press head front face, a peripheral edge and a peripheral outer face,
the peripheral edge is rounded towards the centre of the press head and via a peripheral outer face towards the plinth/plinth wall thereby separating the peripheral outer face from the press head front face,
the press head, with its peripheral edge, is guidable along the anus so that neither the peripheral edge nor the press head front face penetrates into the anal duct and until the peripheral edge or press head to the maximum comes into essentially peripheral pressure contact with the compressor muscle, and
the press head and the base are spaced approx. 30 to 50 mm apart, measured from the upper edge of the press head to the upper edge of base.

2. The device according to claim 1, characterised in that the peripheral edge has a distance/diameter of less than 34 mm, preferably 30 to 22 mm, measured from the opposing peripheral outer faces.

3. The device according to claim 1, characterised in that the plinth comprises a conically reducing and/or cylindrical plinth wall extending from the base towards the press head to reach the peripheral outer face(s).

4. The device according to claim 1, characterised in that the device, with plinth, press head, base in the form of a base plate, consists of or is manufactured from one piece of material.

5. The device according to claim 4, characterised in that the device, with plinth, press head and base plate is deep drawn from the one piece of material.

6. The device according to claim 4, characterised in that the piece of material is plastic or metal and the manufacturing comprises deep drawing.

7. The device according to claim 1, characterised in that the press head is mounted by means of a displaceable and extendable plinth, preferably by means of a plinth exhibiting a threaded rod and a threaded sleeve, in order to separate the base and the press head by a distance ranging from 30 to 50 mm, in particular 35 to 45 mm, measured from the upper edge of the press head to the upper edge of the base.

8. The device according to claim 1, characterised in that the plinth and press head consist of solid material.

9. The device according to claim 1, characterised in that the plinth and/or the press head are round, oval or polygonal with at least 5 rounded corners in relation to a plane of section through the plinth parallel with the standing surface of the base.

10. The device according to claim 1, characterised in that the press head front face is designed convex or concave between the peripheral edges, in particular with a vertical maximum distance of 10 mm, preferably a maximum of 3 mm, from the highest point of the press head front face, in the case of a convex press head front face, or from the lowest point of a concave press head front face, to an imaginary planar surface through the peripheral edge optionally centred in relation to the points of intersection with the peripheral edge.

11. The device according to claim 1, characterised in that the contour shape of the base or base plate is round, rectangular or oval.

12. The device according to at claim 1, characterised in that the device is provided with a bottom cover in the region of the base plate.

13. The device according to claim 12, characterised in that the bottom cover is placed or slid into the base plate or over the base plate.

14. The device according to claim 1, characterised in that the cavity of the device is filled with a coolant in the region of the base and press head, or fitted with a cooling core.

15. The device according to claim 1, characterised in that the rear plinth wall is inclined at an angle of inclination of 2 to 10°, preferably 2 to 5°, from the perpendicular in the forward direction (V)—starting from an upright sitting position of the user.

16. The device according to claim 1, characterised in that the plinth is detachable from the base plate or the base.

17. The device according to claim 1, characterised in that the plinth has a maximum diameter of 34 mm close to the base to 30 mm close to the peripheral outer faces towards the press head, preferably a maximum of 29 mm at the base to 24 mm on the peripheral outer faces.

18. The device according to claim 1, characterised in that the press head front face between the peripheral edges comprises a flattened section which is essentially plane parallel with the standing surface of the base and/or projects up or down from the peripheral edge by no more than 5 mm, in particular by no more than 1 mm.

19. The device according to claim 1, characterised in that the standing surface, in elevation, exceeds the press head front face, in elevation, by more than a factor of 3, preferably by more than a factor of 5.

20. A treatment arrangement comprising a device for repositioning haemorrhoids, comprising a base for stable positioning on an essentially planar stand surface, and a press head supported by a plinth and spaced away from the base and a plane structure placed above the press head, wherein
the device has a length and form ensuring that the press head with the overlaying textile is guidable along the anus to the maximum into the anus until it comes into essentially peripheral pressure contact with the compressor muscle but without penetration of the press head with the overlaying textile into the anal duct, and
the press head and the base are spaced approx. 30 to 50 mm apart, measured from the upper edge of the press head to the upper edge of base.

21. The treatment arrangement according to claim 20 wherein the plane structure is loosely placed above the entire device and/or is selected from the group consisting of a plane film, a plane nonwoven fabric and a worn clothing.

22. The treatment arrangement according to claim 20 wherein
the press head exhibits a press head front face, a peripheral edge and a peripheral outer face and
the peripheral edge is rounded towards the centre of the press head and via a peripheral outer face towards the plinth/plinth wall thereby separating the peripheral outer face from the press head front face,

23. The treatment arrangement according to claim 20 further comprising at least one feature as defined in claim 2.

24. A method for the repositioning of haemorrhoids comprising the following treatment steps
positioning a device with its base protected from falling on an essentially planar stand surface, the device comprising the base for stable positioning and further a press head with a press head surface placed above the press head and a plane surface covering the press head, the device having a length and form ensuring that the press head with the overlaying plane surface is guidable along the anus to the maximum until it comes into essentially peripheral pressure contact with the compressor muscle but without penetration of the press head with the overlaying textile into the anal duct, and the press head and the base are spaced approx. 30 to 50 mm apart, measured from the upper edge of the press head to the upper edge of base,
positioning the anus over the device,
lowering the anus onto the device to guide the press head press head toward the anus until the press head with the overlaying plane surface comes into pressure contact with the compressor muscle.

25. The method of claim 23 wherein the plane structure is loosely placed above the entire device and/or is selected from the group consisting of a plane film, a plane nonwoven fabric and a worn clothing.
